(19) United States
(12) Patent Application Publication
(10) Pub. No.: US 2018/0221217 A1
(43) Pub. Date: Aug. 9, 2018

(54) Maxi Pad With Intergluteal Strip

(71) Applicant: Christina Cuba, Flint, MI (US)

(72) Inventor: Christina Cuba, Flint, MI (US)

(21) Appl. No.: 15/428,101

(22) Filed: Feb. 8, 2017

(51) Int. Cl. A61F 13/475; A61F 13/47; A61F 13/472; A61F 13/505; A61F 13/551; A61F 13/56

(52) U.S. Cl. CPC A61F 13/475; A61F 13/4704; A61F 13/47272; A61F 2013/4587; A61F 13/55135; A61F 13/5616; A61F 13/505

(57) ABSTRACT

An absorbent article for providing protection against menstrual leaks having a pad with an absorbent core and three or more protective layers wherein a middle layer has a deep channel for collecting leakage. The absorbent article also has an adhesive bottom in direct contact with an undergarment and an intergluteal strip that is removably attached to the pad, wherein the intergluteal strip extends away from the distal end, wherein the intergluteal strip is adapted to be disposed within an intergluteal cleft.

### CROSS-REFERENCE TO RELATED APPLICATION(S)

The present application claims priority to U.S. Provisional Patent Application filed on Feb. 8, 2016, entitled “Maxi Pad” the entire disclosure of which is incorporated by reference herein.

### BACKGROUND OF THE INVENTION

### 1. Field of the Invention

The present disclosure relates to absorbent materials and sanitary pads in the field of feminine hygiene products. Further, the present invention solves a issues related to menstrual leaks.

The menstrual cycle is the regular natural change that occurs in the uterus and ovaries that make pregnancy. Up to 80% of women report having some symptoms during the one to two weeks prior to menstruation. Common symptoms include acne, tender breasts, bloating, feeling tired, irritability, and mood changes. These symptoms interfere with normal life and therefore qualify as premenstrual syndrome in 20 to 30% of women. In 3 to 8%, they are severe.

The first period usually begins between twelve and fifteen years of age, a point in time known as menarche. They may occasionally start as early as eight, and this onset may still be normal. The average age of the first period is generally later in the developing world and earlier in developed world. The typical length of time between the first day of one period and the first day of the next is 21 to 45 days in young women and 21 to 31 days in adults (an average of 28 days). Menstruation stops occurring after menopause, which usually occurs between 45 and 55 years of age. Bleeding usually lasts around 2 to 7 days. The bleeding which occurs during menstruation can result increased costs born from steps needed to remedy issues relating to cleanliness larger supplies of clothing, undergarments, pants, and/or bedsheets.

### 2. Description of Related Art

The present disclosure relates to the field of feminine hygiene products. Currently on the market, there are products available to men and women to help with issues relating to spotting, menstrual leaks, and incontinence.

Sanitary pads currently available on the market generally lack the ability to prevent menstrual leaks that normally occur on the back of a woman's underpants or garment. This can be especially embarrassing when sharing a bed with a partner, as the leak can stain the sheets and bed liner, or in a public place, such as school or work, where leaks can stain the back of the pants. Some women may purchase extra long maxi pads, but these pads only extend in length and do not offer a protective barrier that blocks leaks. A suitable solution is desired. Maxi pads can lack wings that make the protective barrier unstable.

Based on the foregoing, there is a need in the art for feminine hygiene devices that protect women from embarrassing and problematic menstrual leaks that occur and may soil or stain women's undergarments, pants, or sheets.

### SUMMARY OF THE INVENTION

The present invention provides superior protection from menstrual leaks over night or during the day and includes a braided tail extension for maximum coverage. The device effectively prevents menstrual leaks from staining a woman's undergarments, pants, or sheets and avoids embarrassment any time of the day. The present invention saves a considerable amount of time and frustration.

The present invention discloses an absorbent article for providing protection against menstrual leaks having a pad with an absorbent core and distal and proximal end. The absorbant pad is composed of three or more protective layers wherein the middle layer has a deep channel for collecting leakage. The pad also has an adhesive bottom in direct contact with a user's undergarment. Overcoming traditional pads in the market, the pad described herein also has an intergluteal strip attached to the distal end of the pad, wherein the intergluteal strip extends away from the distal portion and the intergluteal strip is adapted to be disposed within an intergluteal cleft.

In an embodiment, the intergluteal strip is absorbant, and helps prevent leakage.

In another embodiment, the intergluteal strip is foldable relative to a plane defined by the pad, and is collapsible.

In another embodiment, the intergluteal strip is removeably attached from the distal end of the pad.

In an embodiment, the adhesive bottom contains a plurality of adhesive wings each containing at least one peel away backing having a first layer and a second layer adapted to conform to a wearer's undergarments. The pad can be extended in length.

In an embodiment, the intergluteal strip is woven and braided. Further, the plurality of adhesive wings extends between the proximal end and the distal end along a perimeter of the pad.

In a preferred embodiment, the intergluteal strip is composed of a materials such as rayon, vicose, and cotton. The strip could also be composed of materials which are toxin free. In another embodiment, the absorbant article is biodegradeable. The absorbant article should have at least three or more layers where at least one is an absorbent core. The intergluteal strip further comprises a holding member affixed to the distal end.

Another desired object of the invention herein provides a method for providing protection against a wearer's menstrual leaks. The method comprises the steps of predetermining a configuration for the intergluteal strip wherein the configuration is predetermined based on a user's need, aligning the pad with an undergarment worn by a wearer, attaching the pad to the undergarment wherein an adhesive bottom contacts the undergarment, adjusting the undergarment to the user's body, positioning the intergluteal strip within the user's intergluteal cleft; and removing the at least one peel away backing.

In an embodiment, the method further provides for a step of attaching the pad and folding a plurality of adhesive wings over sides of the undergarments, wherein the adhesive wings secure the pad in place. The step of positioning the intergluteal strip further comprises inserting an intergluteal strip extension between the intergluteal cleft. The absorbant article of claim 12 wherein the holding member acts as the connection between the pad and the intergluteal strip, wherein the holding member can be folded and inserted within a user's intergluteal cleft, and wherein the intergluteal strip can be disassociated from the holding member.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a illustration of the maxi pad and its components, according to an embodiment of the present invention;

FIG. 2 is a standalone view of the device, according to an embodiment of the present invention.

FIG. 3 is a standalone view of the device, according to an embodiment of the present invention.

### DETAILED DESCRIPTION OF PREFERRED EMBODIMENTS

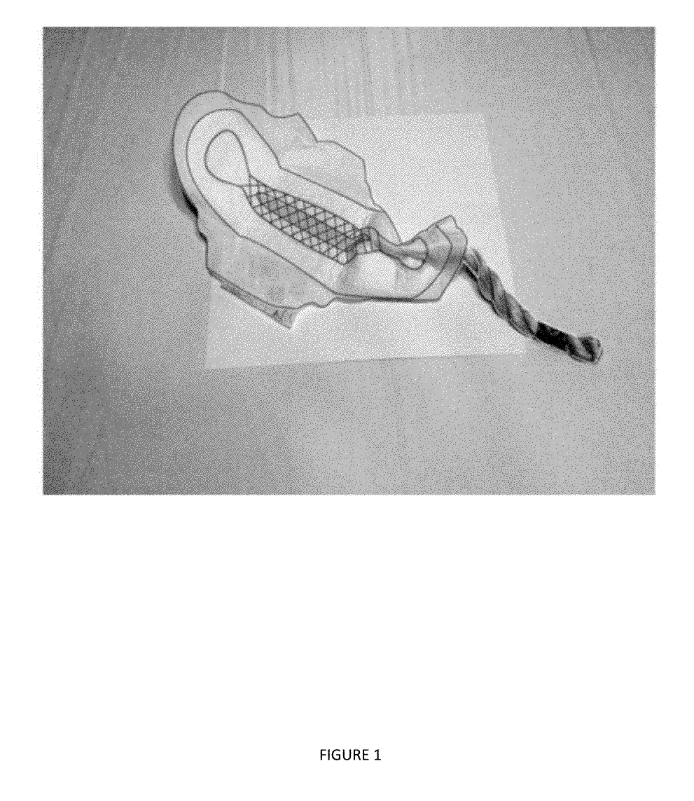

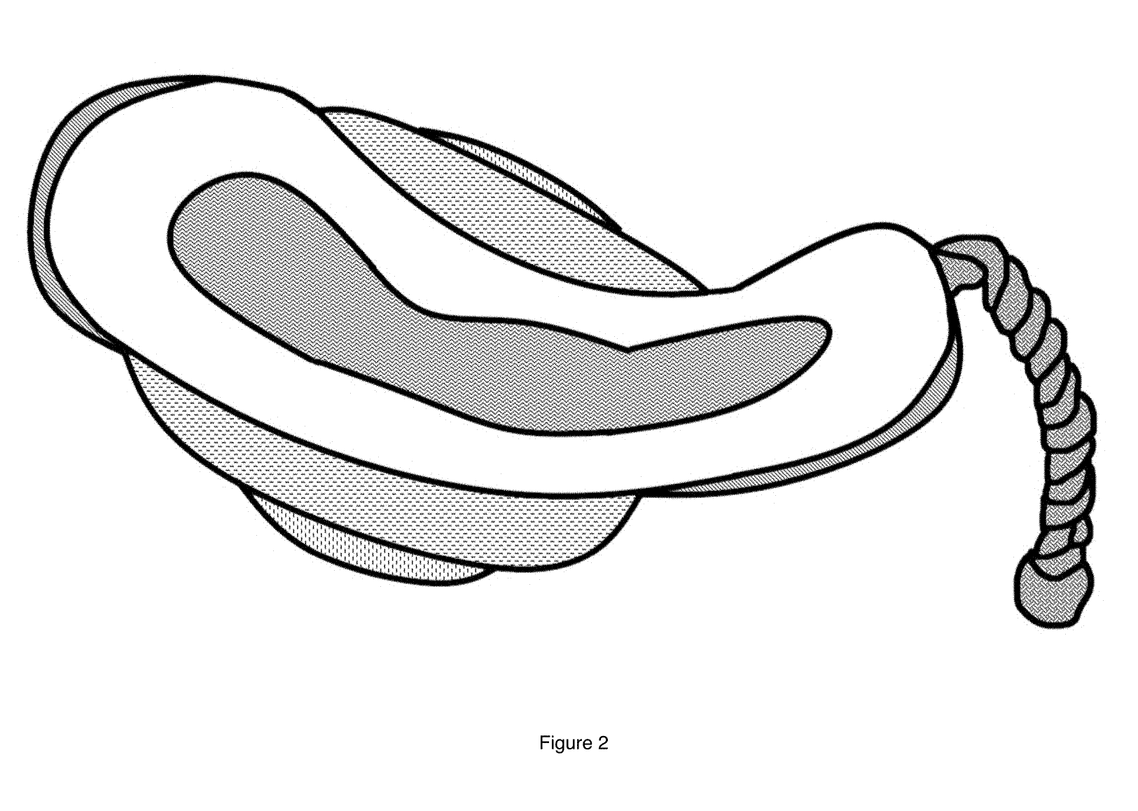

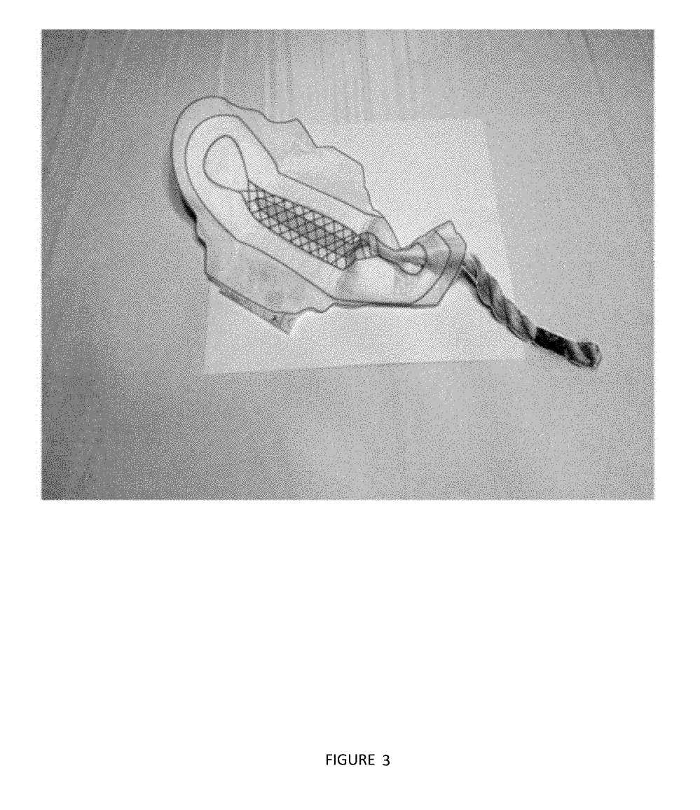

Preferred embodiments of the present invention and their advantages may be understood by referring to FIGS. 1-3, wherein like reference numerals refer to like elements.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

The present invention provides superior protection from menstrual leaks over night or during the day and includes a braided tail extension for maximum coverage. The device effectively prevents menstrual leaks from staining a woman's undergarments, pants, or sheets and avoids embarrassment any time of the day. The present invention saves a considerable amount of time and frustration.

This innovative product may comprise extended-length maxi pad that can be worn day or night during a woman's menstrual cycle. On the sides of the pad can be adhesive wings with a peel-away backing that secure the pad in place. The bottom of the pad can also include an adhesive with a peel-away backing. On the back of the pad can be a woven tail extension. Each pad can be individually wrapped for maximum sanitation.

Instructions:

Unwrap pad and keep wrapper to dispose used pad. Take off bottom wrapper and seal in the center of panties. Remove wing wrappers and seal to the sides of panties. Insert the braided area between butt cheeks as far back as allowed. All steps will prevent leakage throughout the night. Once the braided area has been soiled dispose and replace with a new pad and repeat the following steps.

Maxi Pad is an improved menstrual pad designed to better protect women from leaks during their period. The pad includes an extension for maximum coverage, ensuring all lower-body clothing garments are protected form unwanted stains.

Purpose & Benefits:
1. Provides superior protection from menstrual leaks over night or during the day.
2. Includes a braided tail extension for maximum coverage.
3. Prevents menstrual leaks from staining a woman's undergarments, pant, or sheets.
4. Avoids embarrassment any time of the day.
5. Saves a considerable amount of time and frustration during a period.

Problems Solved:

Most maxi pads currently on the market do not prevent menstrual leaks that normally occur on the back of a woman's undergarment. This can be especially embarrassing when sharing a bed, as the leak can stain the back of the pants. Some women may purchase extra-long maxi pads, but these pads only extend in length and do not offer a protective barrier that fully blocks leaks.

The invention described herein protects women from embarrassing and problematic menstrual leaks. This innovative product may comprise extended-length maxi pad that can be worn day or night during a woman's menstrual cycle. On the sides of the pad can be adhesive wings with a peel-away backing that secure the pad in place. The bottom of the pad can also include an adhesive with a peel-away backing. On the back of the pad can be a woven tail extension. Each pad can be individually wrapped for maximum sanitation.

In use, women can up-wrap the new pad and remove the peel-away backing. They can then press the pad into their panties, and fold the wings over the sides of their panties. They can then pull up their undergarments and insert the woven tail extension between their buttocks as far back as allowed. The woven tail extension can absorb any leakage that attempts to seep through the back of the pad.

The invention can be constructed using comfortable absorbent materials. The pads may be available in a standard size capable of accommodating all women and providing optimal leak coverage. Exact size, measurement, construction, and design specifications may vary upon manufacturing.

In use, women can unwrap the new pad and remove the peel-away backing. They can then press the pad into their panties, and fold he wings over the sides of their panties. They can then pull up their panties and insert the woven tail extension between their butt cheeks, as far back as allowed. The woven tail extension can absorb any leakage that attempts to seep through the back of the pad. The exact specifications may upon manufacturing.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A absorbent article for providing protection against menstrual leaks comprising:
a. an pad having an absorbent core with a distal end and proximal end, wherein the pad comprises:
i. three or more protective layers wherein a middle layer has a deep channel for collecting leakage;
b. an adhesive bottom in direct contact with an undergarment;
c. an intergluteal strip attached to the distal end of the pad, wherein the intergluteal strip extends away from the distal end, wherein the intergluteal strip is adapted to be disposed within an intergluteal cleft.

2. The absorbent article of claim 1, wherein the intergluteal strip is absorbent, wherein the intergluteal strip prevents leakage beyond the distal end.

3. The absorbent article of claim 1, wherein the intergluteal strip is foldable relative to a plane defined by the pad, and wherein the intergluteal strip is collapsible.

4. The absorbent article of claim 1, wherein the intergluteal strip is removeably attached to the distal end.

5. The absorbent article of claim 1, wherein the adhesive bottom further comprises a plurality of adhesive wings each containing at least one peel away backing, having a first layer and a second layer adapted to conform to a wearer's undergarments.

6. The absorbent article of claim one where in the pad is extended length.

7. The absorbent article of claim one wherein the intergluteal strip is woven and braided.

8. The absorbent article of claim two wherein the plurality of adhesive wings extend between the proximal end and the distal end along a perimeter of the pad.

9. The absorbent article of claim one wherein the intergluteal strip is composed of materials selected from the group consisting of: rayon, vicose, and cotton.

10. The absorbent article of claim one wherein the absorbent article is biodegradeable.

11. The absorbent article of claim one wherein the at least one of the three or more layers is an absorbent core.

12. The absorbent article of claim four wherein the intergluteal strip further comprises a holding member affixed to the distal end.

13. The absorbent article of claim 12 wherein the intergluteal strip can vary in dimension, material, length, and width.

14. The absorbent article of claim 13 wherein the holding member acts as the connection between the pad and the intergluteal strip, wherein the holding member can be folded and inserted within a user's intergluteal cleft, and wherein the intergluteal strip can be disassociated from the holding member.

15. A method for providing protection against a wearer's menstrual leaks comprising the steps of:
a. predetermining a configuration for the intergluteal strip wherein the configuration is predetermined based on a user's need;
b. aligning the pad with an undergarment worn by a wearer;
c. attaching the pad to the undergarment wherein an adhesive bottom contacts the undergarment;
d. adjusting the undergarment to the user's body;
e. positioning the intergluteal strip within the user's intergluteal cleft; and
f. removing the at least one peel away backing.

16. The method of claim 14 wherein the step of attaching the pad further comprises:
a. folding a plurality of adhesive wings over sides of the undergarments, wherein the adhesive wings secure the pad in place.

17. The method of claim 15 wherein the step of positioning the intergluteal strip further comprises:
a. inserting an intergluteal strip extension between the intergluteal cleft.
